(19) United States
(12) Patent Application Publication
(10) Pub. No.: US 2013/0245520 A1
(43) Pub. Date: Sep. 19, 2013

(54) SUPPORT DEVICE AND METHODS FOR LIMBS

(71) Applicant: Robert J. Munaretto, Lake in the Hills, IL (US)

(72) Inventor: Robert J. Munaretto, Lake in the Hills, IL (US)

(21) Appl. No.: 13/793,169

(22) Filed: Mar. 11, 2013

Related U.S. Application Data
(60) Provisional application No. 61/610,467, filed on Mar. 13, 2012.

(51) Int. Cl. A61F 5/01

(52) U.S. Cl. CPC A61F 5/0102
USPC 602/6

(57) ABSTRACT

An apparatus for supporting a limb may include an anchoring article having a pre-existing fastening device that includes a hook layer and a loop layer. The anchor article may be worn on a first portion of the limb. The apparatus may also include an elastically deformable band and an anchor coupled proximate to a first end of the band. The anchor is complementary to the pre-existing fastening element and completes a connection in the pre-existing fastening device. The closure device may be coupled to a second end of the band and includes a hook layer and a loop layer. The closure device is configured to fix a first surface of the band to a second surface portion of the band.

### CROSS-REFERENCE TO RELATED APPLICATIONS

This application takes priority from U.S. Provisional Application Ser. No. 61/610,467 filed on Mar. 13, 2012.

### BACKGROUND OF THE DISCLOSURE

1. Field of Disclosure

The present disclosure relates to a devices and methods for supporting human and animal limbs.

2. The Related art

Situations may arise when a human limb may not be able to perform a desired movement or absorb a given loading. In such situations, it is desirable to add some type of external support device to furnish additional strength. The present disclosure relates to devices and methods for providing such additional strength.

### SUMMARY OF THE DISCLOSURE

In aspects, the present disclosure provides an apparatus for supporting a limb. The apparatus may include an anchoring article having a pre-existing fastening device that includes a hook layer and a loop layer, the anchor article being worn on a first portion of the limb; an elastically deformable band sufficiently flexible to wind around at least a second portion of the limb, the band having a length sufficient to wind around the second portion of the limb a plurality of times; an anchor coupled proximate to a first end of the band, the anchor being complementary to the pre-existing fastening element, wherein the anchor completes a connection in the pre-existing fastening device; and a closure device coupled to a second end of the band and including a hook layer and a loop layer, the closure device configured to fix a first surface of the band to a second surface portion of the band, the first and second surfaces being on opposing side of the band.

In aspects, the present disclosure also provides an apparatus for use on a limb in connection with an anchoring article having a pre-existing fastening device. The anchoring article may be worn on a selected location of the limb. The apparatus may include an elastically deformable body formed sufficiently flexible to wind around at least a portion of the limb, the body having a length sufficient to wind around the limb a plurality of times; an anchor coupled to the body, the anchor being complementary to the pre-existing fastening element of the anchoring article, wherein the anchor completes a connection in the pre-existing fastening device; and a closure device coupled to the body, the closure device configured to fix a first portion of the body to a second portion of the body.

The above-recited example of features of the disclosure have been summarized rather broadly in order that the detailed description thereof that follows may be better understood, and in order that the contributions to the art may be appreciated. There are, of course, additional features of the disclosure that will be described hereinafter and which will form the subject of the claims appended hereto.

### BRIEF DESCRIPTION OF THE DRAWINGS

For detailed understanding of the present disclosure, references should be made to the following detailed description of the preferred embodiment, taken in conjunction with the accompanying drawings listed below:

FIG. 1 illustrates a first side of a wrap according to one embodiment of the present disclosure;

FIG. 2 illustrates a second side of a wrap according to one embodiment of the present disclosure; and

FIG. 3 illustrates a portion of a wrap according to one embodiment of the present disclosure connected to an anchor article.

### DETAILED DESCRIPTION OF THE DISCLOSURE

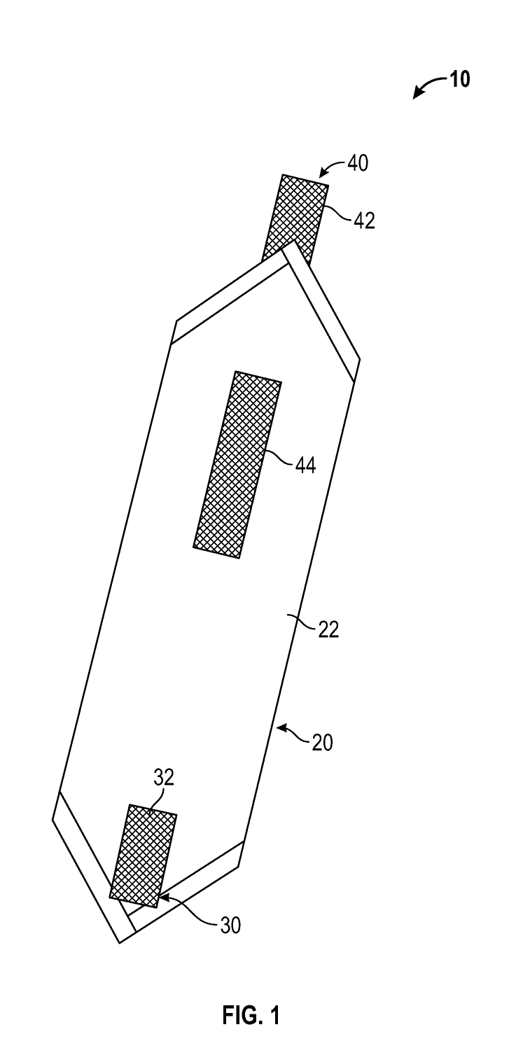

Referring to FIG. 1, there is shown one embodiment of a wrap 10 for supporting a limb while a user performs a desired activity. As used herein, the term “limb” refers to bodily limbs, portions of bodily limbs, and joints along bodily limbs. The wrap 10 may include a body 20, an anchor 30, and a closure device 40. As will be described in greater detail below, the wrap may be initially attached to an article using the anchor 30. The article to which the anchor 20 is attached will be referred to as the “anchor article” or “anchoring article.” Illustrative articles include, but are not limited to, handwear such as gloves, shirts, pants, footwear such as shoes or boots, jackets, belts, suspenders, outerwear, etc. Thereafter, the body 20 is wound around the limb and secured using the closure device 40. It should be appreciated that the anchor 30 prevents undesirable movement of the body 20 as the user applies tension on the body 20 during the winding process.

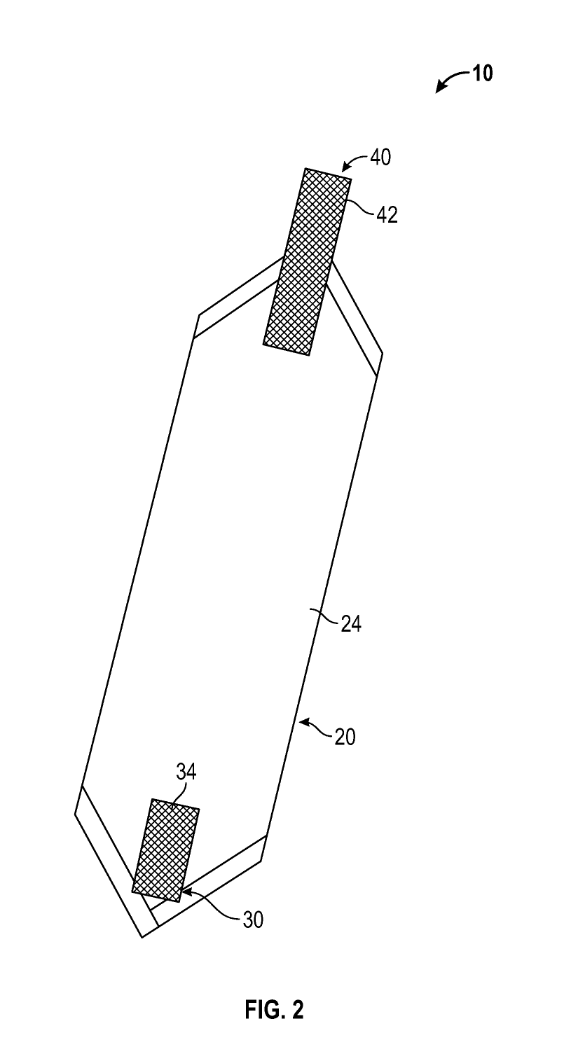

In one embodiment, the body 20 may be an elongated flexible member in the shape of a ribbon, band, sheet, or strip. The body 20 may have a length sufficient to wind around the limb several times. The body 20 has a first side 22 and an opposite second side 24 (FIG. 2). The material making up the body 20 may be natural and/or human-made. The properties of the material will depend on the particular application for the wrap 10. In some embodiments, the body 20 may be formed of a flexible but inelastic material such as cotton, nylon, or leather. In other embodiments, the body 20 may be formed of a flexible and elastic material such as an elastomer (e.g., rubber), stretchable nylon, or spandex. By elastic, it is meant that the material has a functionally effective zone of elastic deformation. By functionally effective, it is mean that the elasticity can be used to perform a desired function, such as generate a compressive force.

The anchor 30 is configured to connect to a portion of the anchor article. In some embodiments, the portion of the anchor article may include a pre-existing fastening device. For example, the fastening device may be a hook layer or a loop layer of a VELCRO-type closure device, a zipper element, a button, a buckle, a clasp, a hook, loops, button holes and other fastening elements used on clothing articles such as a glove, outerwear, footwear, etc. In one arrangement, the anchor 30 is formed complementary to the fastening device of the anchor article. For instance, certain exercise gloves include a VELCRO fastener system. VELCRO fastener systems typically include a loop layer and a hook layer. Therefore, an anchor 30 that is complementary to a VELCRO fastener system may also include a loop layer and a hook layer. For instance, the anchor 30 may have a loop layer 32 (FIG. 1) and a hook layer 34 (FIG. 2). The loop layer 32 (FIG. 1) may on the first body side 22 and a hook layer 34 (FIG. 2) may be on the opposite second body side 24.

Alternatively, the layers 32, 34 may be formed on a tab or other member that extends from the body 20. It should be appreciated that the anchor 30 fastens the body 20 to the anchoring article. This action also connects the two elements of the fastening system of the anchor article. That is, the anchor 30 integrates into and completes the connection of the fastening system of the anchor article. The anchor 30 may be formed at one end of the body 20 as shown or at an intermediate location along between the two ends of the body 20.

In other embodiments, if the anchor article has a button hole, then the complementary anchor 30 may be a button. In still other embodiments, if the anchor article has zipper teeth, then the complementary anchor 30 may be a zipper slider and complementary teeth. It should be understood that these anchor embodiments are merely illustrative and not limiting.

The closure device 40 is configured to secure the body 20 after being wrapped around the limb. By fixing one portion of the body 20 to another portion of the body 20, the closure device 40 maintains the body 20 in a wound position around the wrapped limb. The closure device 40 may be a VELCRO fastening system, a zipper arrangement, a clasp and hook arrangement, a button and buttonhole arrangement, etc. For instance, the closure device 40 may use a VELCRO fastening arrangement that includes a loop layer 42 (FIG. 1) on the first side 22 and a hook layer 44 (FIG. 2) on the second side 24. It should be understood that the layers 42, 44 may be reversed in position. Also, it should be understood that these embodiments are merely illustrative and not limiting. For example, a clasp may be on the first side 22 and a loop may be on the second side 24. The closure device 40 may be formed at one end of the body 20 as shown or at an intermediate location along between the two ends of the body 20.

To describe the use of the wrap 10, reference will be made to a human wrist and hand. Also, for convenience, the anchoring article will be a glove having a VELCRO hook/loop fastening device. It is emphasized, however, that the present disclosure is not limited to any particular limb, whether human or animal, or anchoring article.

The user may have a wrist that requires additional strength as stability in order to perform a desired activity. To use the wrap 10, the user first puts on a glove having a fastening device. Next, the hook layer 32 is attached to the loop layer of the anchor article and the loop layer 34 is attached to the hook layer of the anchor article. It should be noted that by making these attachments, the hook layer and the loop layer of the anchoring article are also connected. Thus, completing the connection between the wrap and the anchoring device also completes the internal connection for the fastening device of the anchor.

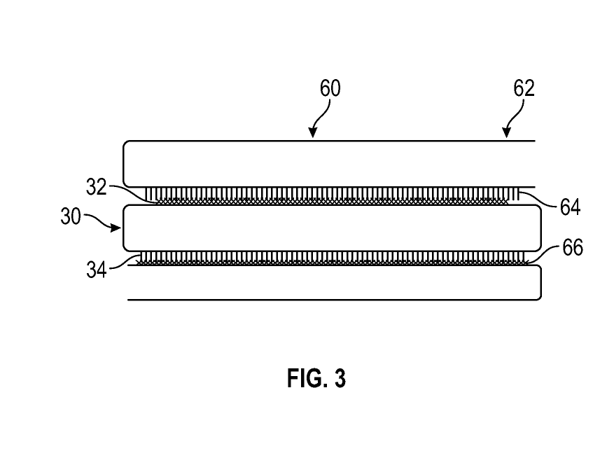

Referring to FIG. 3, there is shown a portion of an anchor article 60 that includes a fastening device 62. The fastening device 62 is a hook and loop arrangement that includes a hook layer 64 and a loop layer 66. The layers 64, 66 of the fastening device 62 may be connected to one another to secure the anchor article 60 to a limb. The anchor 30 includes a complementary hook and loop arrangement, i.e., a loop layer 32 and a hook layer 34. As shown, the anchor 30 interconnects with the fastening device 62 such that a connection is formed within the fastening device 62 and the wrap 10 (FIG. 1) is anchored to the anchor article 60. Thus, the fastening device 62 may be connected to secure the anchor article 60 to the limb at the same time the wrap 10 is connected to the anchor article 60. It should be appreciated that the fastening device 62 is a pre-existing feature that can be activated independent of the anchor 30. That is, the fastening device 62 is structurally complete without the anchor 30 and can independently perform one or specified tasks. The task is predetermined and unrelated to the use of the wrap 10. Illustrative, but not exclusive, tasks including securing the anchoring article to the limb, adjusting a compression, tension, or position of the anchoring article, assisting in the wearing or removing of the anchoring article, etc. For convenience, a pre-existing fastening device 62 that includes the structure to operate independent of the wrap 10 may be referred to as a “functionally independent” feature. For instance, the fastening device 62 may be used in connection with an adjustable band that secures the anchoring article to the limb.

It should be appreciated that using a pre-existing fastening device 62 allows embodiments of the present disclosure to be used with anchor articles that are not specially designed to attach to the wrap 10. Because the anchoring article does not require a special connecting device to attach to the wrap 10, the anchoring article design is simplified. Thus, in an example, a wrap 10 having a hook and loop arrangement may be used with any anchor article that has a complementary hook and loop arrangement.

Because the anchor 30 is connected to the anchor article, one end of the body 20 is secured when tension is applied to the other end of the body 20. Moreover, because the anchor 30 does not directly connect or against to a portion of the limb itself, it should be appreciated that the body 20 can be stretched and pulled without stressing the limb.

Next, the user winds the body 20 around the limb. In embodiment where the body 20 is elastic, the body 20 stretches. The winding will typically result in a multi-layered wrapping around the limb. Upon completion of the wrapping process, the user uses the closure device 40 to secure the wrap 10. For instance, the user may attach the loop layer 42 on the first side 22 to the hook layer 44 on the second side. Thus, the wrap 10 is fixed in a wrapped condition around the limb. Moreover, the body 20, due to elasticity, may apply a compressive force on the wrapped portion of the limb. To unwrap, the user reverses the steps after unfastening the closure device 40.

As noted previously, the wrap is not limited to any particular limb section or anchor article. By way of illustration and not limitation, the wrapping may also be used on elbows by connecting to a shirt sleeve, on knees by connecting to a sock, on an ankle by connecting to a shoe, on a spine region by connecting to a belt, a shoulder by connecting to a shirt, etc. Furthermore, the wrap may be used on animals; e.g., a collar of a dog, reigns of a horse, saddle of a horse, etc.

From the above, it should be appreciated that what has been described includes a stretchable wrap for adding strength and support to limbs and joints such as wrists, elbows, ankles and knees. The wrap may be easily attached to articles such as clothing, gloves, belts, suspenders, outerwear or footwear. The attachment may be using a pre-existing fastening device on the article.

An apparatus for use on a limb in connection with an anchoring article. The apparatus may include an elastically deformable body formed sufficiently flexible to wind around at least a portion of the limb; an anchor coupled to the body, the anchor selectively connecting to the anchor device; and a closure device coupled to the body, the closure device configured to fix a first portion of the body to a second portion of the body. The limb is one of: (i) a portion of a human arm, and (ii) a portion of a human leg. The anchoring article is one of: (i) a handwear, and (ii) footwear.

From the above, it should be appreciated that what has been described includes a wrap that applies a compressive force on a portion of a limb requiring additional strength or support.

From the above, it should be appreciated that what has been described includes a wrap that easily attaches to any glove that has a fastening or closure feature. The fastening or closure feature may be a pre-existing attachment built into the article.

From the above, it should be appreciated that what has been described includes a wrap that can help user those who are injured or suffering from pain. For example, the connection to the anchor articles ensures that the wrap does not obstruct normal movement. Moreover, this external connection eliminates the need to have to wrap around a limb section like a thumb or cover parts of a body, like a palm of a hand.

From the above, it should be appreciated that what has been described includes a wrap that is easy to remove, tighten or loosen but stays secure and locked in place when wrapped and in use.

Possible variations of the expanding/contracting materials would be a leather strap built into a glove that could be wrapped around to add support and limited expansion/contracting. Another variation would be a textile material that would allow for expansion/contraction.

The foregoing description is directed to particular embodiments of the present disclosure for the purpose of illustration and explanation. It will be apparent, however, to one skilled in the art that many modifications and changes to the embodiment set forth above are possible without departing from the scope of the disclosure. Thus, it is intended that the following claims be interpreted to embrace all such modifications and changes.

We claim:

1. An apparatus for supporting a limb, comprising:
an anchoring article having a pre-existing fastening device that includes a hook layer and a loop layer, the anchor article being worn on a first portion of the limb;
an elastically deformable band sufficiently flexible to wind around at least a second portion of the limb, the band having a length sufficient to wind around the second portion of the limb a plurality of times;
an anchor coupled proximate to a first end of the band, the anchor being complementary to the pre-existing fastening element, wherein the anchor completes a connection in the pre-existing fastening device; and
a closure device coupled to a second end of the band and including a hook layer and a loop layer, the closure device configured to fix a first surface of the band to a second surface portion of the band, the first and second surfaces being on opposing side of the band.

2. An apparatus for use on a limb in connection with an anchoring article having a pre-existing fastening device, the anchoring article being worn on a selected location of the limb, comprising:
an elastically deformable body formed sufficiently flexible to wind around at least a portion of the limb, the body having a length sufficient to wind around the limb a plurality of times;
an anchor coupled to the body, the anchor being complementary to the pre-existing fastening element of the anchoring article, wherein the anchor completes a connection in the pre-existing fastening device; and
a closure device coupled to the body, the closure device configured to fix a first portion of the body to a second portion of the body.

3. The apparatus of claim 1, wherein the anchor is a hook and loop fastening device.

4. The apparatus of claim 3, wherein the anchor includes a hook layer and a loop layer.

5. The apparatus of claim 4, wherein the hook layer and the loop layer are on opposingly arranged.

6. The apparatus of claim 5, wherein the anchor is interleaved with the fastening device of the anchor article when connected to the fastening device of the anchor article.

7. The apparatus of claim 2, wherein the closure device is a hook and loop fastening device.

8. The apparatus of claim 7, wherein the closure device includes a hook layer and a loop layer.

9. The apparatus of claim 8, wherein the hook layer and the loop layer are on opposingly arranged.

10. The apparatus of claim 2, wherein the body is formed as a band, wherein the anchor is formed proximate to a first end of the band and the closure device is formed proximate to a second end of the band.
